(19) United States
(12) Patent Application Publication
(10) Pub. No.: US 2021/0356282 A1
(43) Pub. Date: Nov. 18, 2021

(54) INFORMATION PROCESSING SYSTEM, HEALTH MANAGEMENT SYSTEM, COMPUTER-READABLE STORAGE MEDIUM AND INFORMATION PROCESSING METHOD

(71) Applicant: HONDA MOTOR CO.,LTD., Tokyo (JP)

(72) Inventors: Kazunori TOKUNAGA, Tokyo (JP); Mafuyu KOSEKI, Tokyo (JP); Wataru AIKO, Tokyo (JP); Toshiaki TAKANO, Tokyo (JP); Satoshi ONODERA, Tokyo (JP)

(21) Appl. No.: 17/385,956

(22) Filed: Jul. 27, 2021

Related U.S. Application Data
(63) Continuation of application No. PCT/JP2019/003755, filed on Feb. 1, 2019.

(51) Int. Cl. G01C 21/34; G01C 21/36

(52) U.S. Cl. CPC G01C 21/3423; G01C 21/3617

(57) ABSTRACT

An information processing system comprises: a first destination information acquisition section for acquiring a first destination information indicating a first destination which is a destination of a user; a physical information acquisition section for acquiring a physical information indicating a physical feature of the user; a candidate point determination section for determining a candidate point of a second destination which is a destination of a moving body for conveying the user, based on (i) a position of the first destination indicated by the first destination information, and (ii) a physical feature of the user indicated by the physical information. The first destination and the second destination are different from each other.

The contents of the following international application are incorporated herein by reference:

NO. PCT/JP2019/003755 filed in WO on Feb. 1, 2019.

### BACKGROUND

### 1. Technical Field

The present invention relates to an information processing system, health management system, a computer-readable storage medium and an information processing method.

### 2. Related Art

A system for searching a pathway from the start point to the destination is known (for example, see Patent Documents 1 to 2).

### Patent Document

[Patent Document 1] Japanese Patent Unexamined Publication No. 2018-081022

[Patent Document 2] Japanese Patent Unexamined Publication No. 2018-155498

### BRIEF DESCRIPTION OF THE DRAWINGS

FIG. 1 schematically shows an example of a system configuration of a navigation system 100.

FIG. 2 schematically shows an example of an interior configuration of a vehicle 20.

FIG. 3 schematically shows an example of an interior configuration of a control section 260.

FIG. 4 schematically shows an example of an interior configuration of an assistance server 120.

FIG. 5 schematically shows an example of an interior configuration of a storage section 440.

FIG. 6 schematically shows an example of an interior configuration of a navigation assistance section 450.

FIG. 7 schematically shows an example of an interior configuration of a candidate point determination section 632.

FIG. 8 schematically shows an example of an interior configuration of a control event detection section 460 and a control instruction generation section 470.

FIG. 9 schematically shows an example of an interior configuration of a control section 960.

FIG. 10 schematically shows an example of a system configuration of a computer 3000.

### DESCRIPTION OF EXEMPLARY EMBODIMENTS

Hereafter, the present invention will be explained through embodiments of the inventions, however, the following embodiments do not limit the inventions according to the claims. Further, not all the combinations of the features explained in the embodiments are necessarily essential for the means for solving the problems of the inventions. Note that, in the figures, the same reference numerals may be assigned to the same or similar portions so as to omit repetitive explanations in some cases.

[Outline of the Navigation System 100]

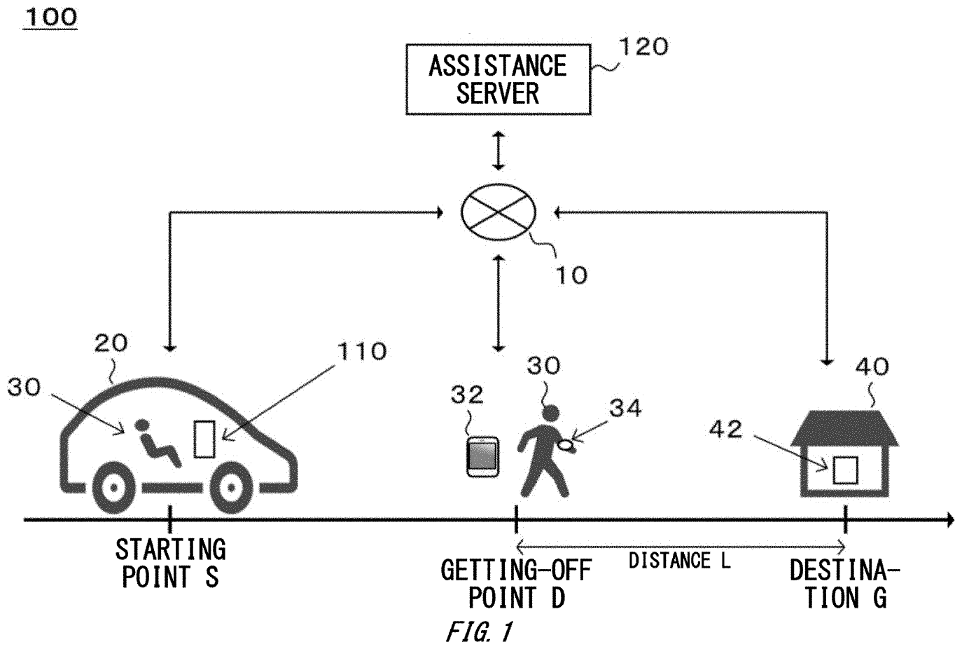

FIG. 1 schematically shows an example of a system configuration of a navigation system 100. In this embodiment, the navigation system 100 comprises a vehicle control system 110 mounted to a vehicle 20 and an assistance server 120. The vehicle control system 110 and the assistance server 120 may send and receive information via a communication network 10 to/from each other. Each of the vehicle control system 110 and the assistance server 120 may send and receive information between itself and a communication terminal 32 of a user 30 who utilizes the vehicle 20.

In this embodiment, the navigation system 100 determines a driving route of the vehicle 20. For example, if a start point S and a destination G of the user 30 are inputted, the navigation system 100 sets a point D which is geographically different from the destination G of the user 30 (this is called “getting-off point” in some cases) as the destination of the vehicle 20. The navigation system 100 may determine the driving route of the vehicle 20, based on the start point S of the user 30 and the getting-off point D of the user 30.

When the vehicle 20 arrives at the getting-off point D, the user 30 gets off the vehicle 20. Thereafter, the user 30 moves from the getting-off point D to the destination G by means of the physical activity of the user 30, without utilizing the vehicle 20. As the physical activity of the user 30, a walking, a rapid walking, a jogging, a running, a cycling and the like are exemplified.

In this embodiment, the getting-off point D is determined based on (i) the position of the destination G of the user 30, and (ii) the physical feature of the user 30. For example, a condition for a distance L between the destination G and the getting-off point D is determined based on (i) the position of the destination G of the user 30, and (ii) the physical feature of the user 30. As the physical feature of the user 30, at least one of a gender, an age, a body height, a body weight, a muscle mass, a body fat percentage, an ideal body weight, an ideal muscle mass, an ideal body fat percentage, a current health state, a presence/absence of a disorder or an injury, a level of a disorder or an injury, a clinical history, a medical history, a presence/absence of an exercise experience, and a level of an exercise experience are exemplified.

In this embodiment, the getting-off point D is determined, for example, based on (i) a position of the destination G of the user 30, and (ii) a target value of an energy equivalent amount to be consumed if the user 30 moves from the getting-off point D to the destination G. As the energy equivalent amount, at least one of a calorie consumption amount, a fat burning amount, a carbohydrate burning amount and a physical activity amount is exemplified. The energy equivalent amount may be an index calculated by using at least one of the calorie consumption amount, the fat burning amount, the carbohydrate burning amount and the physical activity amount.

The energy consumed when the user 30 moves from the getting-off point D to the destination G fluctuates depending on a physical feature of the user 30. As such, the navigation system 100 determines the position of the getting-off point D, for example, based on (i) the position of the destination G of the user 30, (ii) the target value of the energy equivalent amount to be consumed if the user 30 moves from the getting-off point D to the destination G, and (iii) the physical feature of the user 30. By this, the navigation system 100 can determine an appropriate position as the getting-off point D, with a comparatively small amount of calculation.

The above-described target value of the energy equivalent amount may be determined based on an input by the user 30. For example, the user 30 inputs an energy equivalent amount, which the user 30 desires to consume during the period until the user 30 arrives to the destination G, to the communication terminal 32 or the vehicle control system 110. The user 30 may input an energy equivalent amount which the user 30 desires to consume during the period when the user 30 reciprocates between the start point S and the destination G. The user 30 may input a category of the physical activity. For example, the user 30 inputs the information indicating that said user desires to consume calories of 200 kcal by walking during the period until he/she arrives to the destination G, to the communication terminal 32 or the vehicle control system 110.

The user 30 may input a target related to a diet or a training to the communication terminal 32 or the vehicle control system 110. The above-described target may include the information indicating the ideal value related to at least one of a body weight, a muscle mass and a body fat percentage. The above-described target may include the information indicating the ideal value related to at least one of an information indicating a time limit of a diet or a training, and a body weight, a muscle mass and a body fat percentage at the time of said time limit. For example, the target value of the above-described energy equivalent amount is determined so as to attain the target related to the diet or the training.

The target value of the above-described energy equivalent amount may be determined based on the physical feature of the user 30. For example, the target value of the above-described energy equivalent amount is determined based on the difference between the current value and its ideal value, of at least one of the body weight, the muscle mass and the body fat percentage of the user 30.

The target value of the above-described energy equivalent amount may be determined based on the category of a facility 40 positioned in the destination G of the user 30, or the category of the service provided in the facility 40. For example, the target value of the above-described energy equivalent amount is determined based on the calorie amount of a meal provided in the facility 40.

For example, if the facility 40 is a restaurant, the getting-off point D will be determined so that the distances to the getting-off point D and the destination G should be in accordance with the content of the meal provided in said facility. Further, if the facility 40 is a training gym, the getting-off point D may be determined so that the distances to the getting-off point D and the destination G should be appropriate for warming-up or cooling-down.

As described above, according to the navigation system 100 according to this embodiment, distances to the getting-off point D and the destination G are appropriately adjusted based on the status of the user 30. By this, the navigation system 100 can manage the health of the user 30. Also, the navigation system 100 according to this embodiment can determine an appropriate position as the getting-off point D with a comparatively small amount of calculation.

[Outline of Each Portion of the Navigation System 100]

In this embodiment, the communication network 10 may be a transmission path of a wired communication, or may be a transmission path of a wireless communication, or may be a combination of a transmission path of a wired communication and a transmission path of a wireless communication. The communication network 10 may include a wireless packet communication network, an Internet, a P2P network, a dedicated line, a VPN, a power line communication line or the like. The communication network 10: (i) may include a mobile communication network such as a mobile phone line network; or (ii) may include a wireless data communication network such as a wireless MAN (for example, WiMAX (registered trademark)), a wireless LAN (for example, WiFi (registered trademark)), a Bluetooth (registered trademark)), a Zigbee (registered trademark)), or an NFC (Near Field Communication).

In this embodiment, the vehicle 20 conveys the user 30. The vehicle 20 may move by means of manipulation by a driver, or may move by means of automatic operation or remote manipulation. The vehicle 20 may be a vehicle which moves pursuant to a predefined operation plan, or may be a vehicle which moves in accordance with a request of the user 30. The operation plan defines multiple stations and/or stops for the vehicle 20 to stop, and a scheduled time for stopping at each station or each stop. Positions of the stations and/or stops are predefined, and principally, they cannot be changed in accordance with a request by the user 30.

As the vehicle to move pursuant to a predefined operation plan, a route bus, a long-distance bus, an omnibus, a railcar, a passenger aircraft or the like are exemplified. The vehicle to move pursuant to the request of the user 30 may be a vehicle reserved by the user 30, or a vehicle reserved by comparatively small number of crew or passengers including the user 30. As the vehicle to move pursuant to the request by the user 30, a taxi, a rental car, a shared car, a vehicle provided for use of ride sharing and the like are exemplified. As the vehicle 20, an automobile, a motorcycle, an electric train and the like are exemplified. As the automobile, an engine vehicle, an electric vehicle, a fuel cell vehicle, a hybrid vehicle, a work machine and the like are exemplified. As the motorcycle, (i) a motorbike, (ii) a three-wheeled motorcycle, and (iii) a standing ride bicycle or tricycle equipped with a power unit, and the like, are exemplified.

Note that, if the vehicle 20 is a vehicle that moves pursuant to a predefined operation plan, the getting-off point D is selected among multiple stations and/or stops for the vehicle 20 to stop. In this case, the navigation system 100 selects, for example, a station or a stop, whose absolute value of the difference between the energy equivalent amount to be consumed if the user 30 moves along the shortest pathway from said station or stop to the destination G and the target value of the energy equivalent amount to be consumed if the user 30 moves from the getting-off point D to the destination G is smaller than the predefined value, as the getting-off point D, among multiple stations and/or stops existing in the vicinity of the destination G.

If there is no station or stop whose absolute value of the above-described difference is smaller than the predefined value, the navigation system 100 selects, for example, a station or stop, whose absolute value of the above-described difference is the smallest, as the getting-off point D. In this case, the navigation system 100 may select a station or a stop, whose absolute value of the above-described difference is the smallest, among the stations and/or stops, of which the energy equivalent amount to be consumed if the user 30 moves along the shortest pathway from said station or stop to the destination G is greater than the target value of the energy equivalent amount to be consumed if the user 30 moves from the getting-off point D to the destination G, as the getting-off point D.

If there is no station or stop whose absolute value of the above-described difference is smaller than the predefined value, the navigation system 100 may select a station or a stop, whose absolute value of the above-described difference is the smallest, among the stations and/or stops, of which the energy equivalent amount to be consumed if the user 30 moves along the shortest pathway from said station or stop to the destination G is smaller than the target value of the energy equivalent amount to be consumed if the user 30 moves from the getting-off point D to the destination G, as the getting-off point D. The navigation system 100 may determine a moving pathway from the station or stop selected as the getting-off point D to the destination G, based on the target value of the energy equivalent amount to be consumed if the user 30 moves from the getting-off point D to the destination G. In this case, the distance of the above-described moving pathway is longer than the distance of the shortest pathway from the getting-off point D to the destination G.

In this embodiment, the user 30 moves utilizing the vehicle 20. The user 30 may carry a communication terminal 32. The user 30 may carry a wearable terminal 34.

In this embodiment, the communication terminal 32 sends and receives various kinds of information between itself and at least one of the vehicle control system 110 and the assistance server 120. For example, the communication terminal 32 sends and receives various kinds of information between itself and at least one of a vehicle control system 110 and an assistance server 120, via a communication network 10. The communication terminal 32 may send and receive various kinds of information between itself and the vehicle control system 110, by means of P2P communication. The communication terminal 32 may have an input device and an output device so as to be utilized as the user interface of the navigation system 100.

The communication terminal 32 may collect a vital information of the user 30 from the wearable terminal 34 worn by the user 30. As the vital information, a respiratory state, a pulse rate, a heart rate, a blood pressure, a blood oxygen concentration, a blood glucose, a blood triglyceride level, a blood cholesterol level, a body temperature, a perspiration state, a gastrointestinal state, an utterance state, an excited state, a wakefulness state and the like are exemplified. The communication terminal 32 may send the vital information of the user 30 to at least one of the vehicle control system 110 and the assistance server 120.

The communication terminal 32 is only required to be an information processing apparatus that can be utilized by the user 30, and its detail is not particularly limited. As the communication terminal 32, a personal computer, a mobile terminal and the like are exemplified. As the mobile terminal, a mobile phone, a smartphone, a PDA, a tablet, a notebook computer or a laptop computer, a wearable computer and the like are exemplified.

In this embodiment, the wearable terminal 34 may have a vital sensor to collect the vital information of the user 30. The wearable terminal 34 may send the collected vital information to the communication terminal 32.

The wearable terminal 34 is only required to be an information processing apparatus to be worn by the user 30 on his/her body, and its detail is not particularly limited. The wearable terminal 34 may be worn by the user 30 to his/her exterior. The wearable terminal 34 is worn, for example, on a head, a breast, an abdomen, a wrist or an ankle, or a finger or toe of the user 30. Apart or all of the wearable terminal 34 may be implanted in the body of the user 30.

In this embodiment, the facility 40 is positioned at the destination G of the user 30. In the facility 40, for example, purchase and sale of a goods or provision of a service is performed. In the facility 40, an activity history acquisition section 42 may be disposed.

The activity history acquisition section 42 acquires an activity history information in which an activity of the user 30 in the facility 40 is recorded. The activity history acquisition section 42 acquires, for example, an information indicating at least one of the category of the goods purchased by the user 30 and the category of the service utilized by the user 30. As the activity history acquisition section 42, an order receiving machine, a payment processing terminal, an entry/exit management machine and the like are exemplified. As the payment processing terminal, a checkout machine, a POS terminal, a credit card payment processing terminal and the like are exemplified.

The activity history acquisition section 42 may send the activity history information to at least one of the vehicle control system 110 and the assistance server 120. The activity history acquisition section 42 may send the activity history information to the communication terminal 32.

In this embodiment, the vehicle control system 110 is mounted to the vehicle 20. The vehicle control system 110 controls the action of the vehicle 20. The vehicle control system 110 may control the action of the vehicle 20 pursuant to the manipulation by the user 30. The vehicle control system 110 may have an automatic operation function or a remote operation function. The vehicle control system 110 may control the action of the vehicle 20 pursuant to the instruction from the assistance server 120.

The vehicle control system 110 may send an information indicating the position of the vehicle 20 to the assistance server 120. The vehicle control system 110 may send an information indicating the state of the vehicle 20 to the assistance server 120. The vehicle control system 110 may acquire a physical information of the user 30. The vehicle control system 110 may send a physical information of the user 30 to the assistance server 120. The vehicle control system 110 may acquire a vital information of the user 30. The vehicle control system 110 may send a vital information of the user 30 to the assistance server 120.

The vehicle control system 110 may determine a driving route for the vehicle 20. For example, the vehicle control system 110 determines the driving route for the vehicle 20 by means of setting the destination of the vehicle 20, based on the physical information of the user 30. Detail of the vehicle control system 110 will be described later.

In this embodiment, the assistance server 120 assists searching a driving route for the vehicle 20. For example, the assistance server 120 acquires the physical information of the user 30 from at least one of the communication terminal 32 and the vehicle control system 110. The assistance server 120 may store the physical information of the user 30 in any storage apparatus. The assistance server 120 determines at least a part of the driving route for the vehicle 20, based on the physical information of the user 30. The assistance server 120 may determine at least a part of the driving route for the vehicle 20, based on the request from the vehicle control system 110.

For example, the assistance server 120 acquires an information indicating the start point S and the destination G of the user 30 from the vehicle control system 110. The assistance server 120 determines the candidate point for the getting-off point D, based on (i) the position of the destination G of the user 30, and (ii) the physical feature of the user 30. The assistance server 120 may determine a single point as the candidate point for the getting-off point D, or may determine multiple points as the candidate points for the getting-off point D.

The assistance server 120 may send an information indicating the candidate point for the getting-off point D to the vehicle control system 110. Further, the assistance server 120 may make the vehicle control system 110 execute a procedure to output an information indicating the candidate point for the getting-off point D to the user 30 and a procedure to acquire the information indicating the candidate point selected as the getting-off point D by the user 30. The assistance server 120 may also make the vehicle control system 110 execute a procedure to send an information indicating the candidate point selected by the user 30 to the assistance server 120.

The assistance server 120 may acquire an information indicating the candidate point selected by the user 30 from the vehicle control system 110 to determine the getting-off point D. The assistance server 120 may determine the driving route for the vehicle 20, based on the positions of the start point S and the getting-off point D. The assistance server 120 may send the information indicating the driving route for the vehicle 20 to the vehicle control system 110. The assistance server 120 may determine the moving route for the user 30, based on the positions of the getting-off point D and the destination G. The assistance server 120 may send an information indicating the moving route for the user 30 to at least one of the vehicle control system 110 and the communication terminal 32.

The assistance server 120 may remotely manipulate the vehicle 20. For example, if the occurrence of an event of a specific category is detected, the assistance server 120 sends an instruction for remotely manipulating the vehicle 20 (this is called “control instruction” in some cases) to the vehicle control system 110. Detail of the assistance server 120 will be described later.

The vehicle 20 may be an example of a moving body. The destination of the user 30 may be an example of a first destination. The getting-off point of the user 30 may be an example of a second destination. The communication terminal 32 may be an example of a physical information acquisition section and a vital information acquisition section. The wearable terminal 34 may be an example of the vital information acquisition section. The navigation system 100 may be an example of an information processing system and a health management system. The vehicle control system 110 may be an example of the physical information acquisition section, the vital information acquisition section and the vehicle control section. The assistance server 120 may be an example of the information processing system, the health management system, the physical information acquisition section, the vital information acquisition section and a user profile management section.

Note that, in this embodiment, detail of the navigation system 100 is explained, taking a case where the assistance server 120 determines the getting-off point for the user 30 as an example. However, the navigation system 100 is not limited to this embodiment. In another embodiment, at least a part of the assistance server 120 may be realized by the vehicle control system 110. In this case, the vehicle control system 110 may be an example of the information processing system, the health management system and the user profile management section. At least a part of the functions of the assistance server 120 may be realized by the communication terminal 32. In this case, the communication terminal 32 may be an example of the information processing system, the health management system, the vehicle control section and the user profile management section.

Note that, in this embodiment, detail of the navigation system 100 is explained, taking a case where the navigation system 100 determines a destination or its candidate point for the vehicle 20 as an example. However, the navigation system 100 is not limited to this embodiment. In another embodiment, the navigation system 100 may determine a destination or its candidate point of the moving body of any category, similarly to the case of the vehicle 20.

As the moving body, a vehicle, a marine vessel, a flying object and the like are exemplified. As the vehicle, an automobile, a motorcycle, an electric train and the like are exemplified. As the automobile, an engine vehicle, an electric vehicle, a fuel cell vehicle, a hybrid vehicle, a work machine and the like are exemplified. As the motorcycle, (i) a motorbike, (ii) a three-wheeled motorcycle, and (iii) a standing ride bicycle or tricycle equipped with a power unit, and the like, are exemplified. As the marine vessel, a ship, a hovercraft, a water bike, a submarine, a submersible craft, an underwater scooter and the like are exemplified. As the flying object, an air plane, an air ship or a balloon, a hot-air balloon, a helicopter, a drone and the like are exemplified.

[Concrete Configuration of Each Portion of the Navigation System 100]

Each portion of the navigation system 100 may be realized by means of a hardware, may be realized by means of a software, or may be realized by means of a hardware and a software. As for each portion of the navigation system 100, at least a part thereof may be realized by a single server, or may be realized by multiple servers. As for each portion of the navigation system 100, at least a part thereof may be realized on a virtual server or a cloud system. As for each portion of the navigation system 100, at least a part thereof may be realized by means of a personal computer or a mobile terminal. As the mobile terminal, a mobile phone, a smartphone, a PDA, a tablet, a notebook computer or a laptop computer, a wearable computer and the like can be exemplified. Each portion of the navigation system 100 may store an information, utilizing a distributed register technology or a distributed network, such as a block chain.

If at least a part of the components constituting the navigation system 100 is realized by means of a software, the component to be realized by said software may be realized, in an information processing apparatus with a general configuration, it may be realized by means of activating a program which defines an action related to said component. The above-described information processing apparatus with a general configuration may comprise (i) a data processing apparatus having a processor such as a CPU or a GPU, or a ROM, a RAM, a communication interface and the like, (ii) an input apparatus such as a keyboard, a pointing device, a touch panel, a camera, a voice/sound input apparatus, a gesture input apparatus, various kinds of sensors, a GPS receiver, and (iii) an output apparatus such as a display apparatus, a voice/sound output apparatus, a vibration apparatus, and (iv) a storage apparatus (including an external storage apparatus) such as a memory, HDD, SSD and the like.

In the above-described information processing apparatus with a general configuration, the above-described data processing apparatus or storage apparatus may store the above-described program. The above-described program is executed by a processor to thereby make the above-described information processing apparatus execute actions defined by said program.

The above-described program may be stored in a non-transitory computer-readable storage medium. The above-described program may be stored in a computer-readable medium such as a CD-ROM, a DVD-ROM, a memory, a hard disk, or may be stored in a storage apparatus connected to a network.

The above-described program may be a program for making the computer function as the navigation system 100 or as a part thereof. The above-described program may include a module which defines the action of each portion of the navigation system 100. This program or module coerces on the data processing apparatus, input apparatus, output apparatus, storage apparatus, etc., to accordingly make the computer function as each portion of the navigation system 100, or make the computer execute an information processing method at each portion of the navigation system 100. The above-described program may be installed to a computer which configures at least a part of the navigation system 100 from a storage apparatus connected to a computer-readable medium or a network. The above-described program may be executed to thereby make the computer function as at least a part of each portion of the navigation system 100.

Information processing described in the above-described program will, as said program is read by the computer, thereby function as a concrete means, in which the software related to said program and the navigation system 100 or various kinds of hardware resources as a part thereof cooperate. And thus, the above-described concrete means realizes computation or processing of information corresponding to the purpose of use of the computer in this embodiment, thereby the navigation system 100 corresponding to said purpose of use will be established.

The above-described program may be a program for making the computer function as the vehicle control system 110. The above-described program may be a program for making the computer execute an information processing method in the vehicle control system 110. The above-described program may be a program for making the computer function as the assistance server 120. The above-described program may be a program for making the computer execute an information processing method in the assistance server 120.

The above-described information processing method has, for example, a first destination information acquisition step for acquiring a first destination information indicating a first destination which is the destination of the user. The above-described information processing method has, for example, a physical information acquisition step for acquiring a physical information indicating a physical feature of the user. The above-described information processing method has, for example, (i) a position of the first destination indicated by the first destination information, and (ii) a candidate point determination step for determining a candidate point for the second destination which is the destination of the moving body carrying the user based on the physical feature of the user indicated by the physical information. In the above-described information processing method, the first and second destinations may be geographically different from each other.

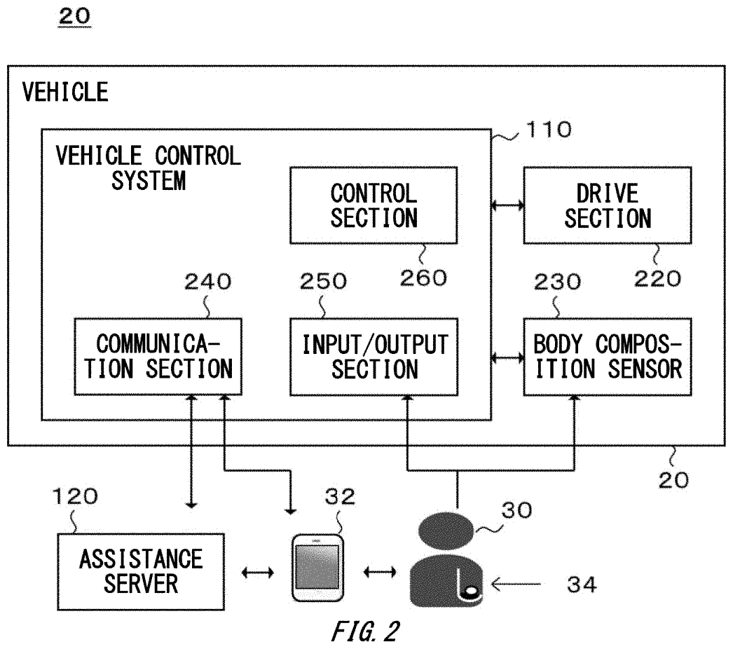

FIG. 2 schematically shows an example of an interior configuration of a vehicle 20. In this embodiment, the vehicle 20 includes a vehicle control system 110, a drive section 220, and a body composition sensor 230. In this embodiment, the vehicle control system 110 includes a communication section 240, an input/output section 250, and a control section 260.

In this embodiment, the drive section 220 drives the vehicle 20. The drive section 220 may move the vehicle 20 pursuant to the control by the vehicle control system 110.

In this embodiment, the body composition sensor 230 acquires an information indicating the body composition of the user 30. As the body composition, a body weight, a body fat percentage, a body moisture percentage, a muscle mass, a bone mineral density and the like are exemplified. The body composition sensor 230 may output the information indicating the body composition of the user 30 to the vehicle control system 110.

The body composition sensor 230 may, for example, output an estimated value of the body weight of the user 30, based on the output of the pressure meter positioned at the seat of the vehicle 20. The body composition sensor 230 may output an estimated value of at least one of the body fat percentage, the body moisture percentage, the muscle mass and the body mineral density of the user 30, based on the output of a pair of electrodes positioned at the handle of the vehicle 20 and the information indicating the gender and the age of the user 30. The body composition sensor 230 may, for example, acquire an information indicating the gender and the age of the user 30, from the vehicle control system 110.

In this embodiment, the communication section 240 establishes communication between itself and at least one of the communication terminal 32 and the assistance server 120, to thereby send and receive various kinds of information. The communication section 240 may be compliant to one or more communication system.

In this embodiment, the input/output section 250 receives an input or a command from the user 30. The input/output section 250 outputs the information to the user 30. The input/output section 250 may have various kinds of input apparatus, such as a keyboard, a touch panel, a pointing device, a camera, a microphone, various kinds of sensors, a GPS receiver and the like. The input/output section 250 may have a voice/sound input system. The input/output section 250 may have a gesture input system. The input/output section 250 may have various kinds of output apparatus, such as a display apparatus, a lighting apparatus, a speaker, a vibration apparatus, a vehicle body raising/lowering apparatus and the like.

In this embodiment, the control section 260 controls the action of the vehicle 20. The control section 260 may receive a command from the user 30 to control the action of the vehicle 20 pursuant to the command from the user 30. The control section 260 may control the action of the vehicle 20 by means of an automatic operation function or a remote operation function. The control section 260 may receive an instruction from the assistance server 120 to thereby control the action of the vehicle 20 pursuant to the instruction from the assistance server 120. Detail of the control section 260 will be described later.

The body composition sensor 230 may be an example of the physical information acquisition section. The communication section 240 may be an example of the first destination information acquisition section, the category information acquisition section, the mode information acquisition section, the time information acquisition section, the road information acquisition section, the selection information acquisition section and the vital information acquisition section. The input/output section 250 may be an example of the first destination information acquisition section, the category information acquisition section, the mode information acquisition section, the time information acquisition section, the road information acquisition section and the selection information acquisition section. The control section 260 may be an example of the physical information acquisition section, the vital information acquisition section and the vehicle control section.

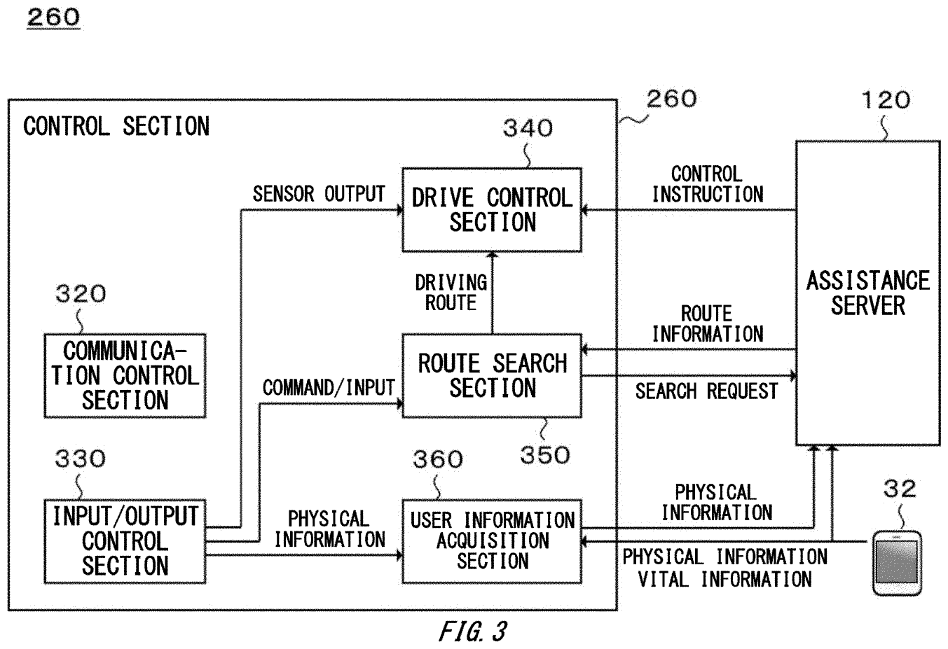

FIG. 3 schematically shows an example of an interior configuration of a control section 260. In this embodiment, the control section 260 includes a communication control section 320, an input/output control section 330, a drive control section 340, a route search section 350 and a user information acquisition section 360.

In this embodiment, the communication control section 320 controls the communication section 240. The communication control section 320 may control the communication between each portion of the control section 260 and at least one of the communication terminal 32 and the assistance server 120. The communication control section 320 may be compliant to one or more communication system.

In this embodiment, the input/output control section 330 controls the input/output section 250. In one embodiment, the input/output control section 330 may control the display apparatus of the input/output section 250 to output an input screen or a selection screen to be utilized as the user interface of the navigation system 100. The input/output control section 330 may acquire an information or a command inputted on the input screen or the selection screen by the user 30. In another embodiment, the input/output control section 330 may control the speaker of the input/output section 250 to output a voice/sound of a guide information. The guide information may be an information for facilitating the user 30 to input an information or a command. The input/output control section 330 may analyze the voice/sound of the user 30 to acquire an information or a command inputted by the user 30.

In another embodiment, the input/output control section 330 may control the input/output section 250 to output various kinds of alerts to the user 30. For example, if the user 30 gets off the vehicle 20 with the communication terminal 32 left in the vehicle 20, the input/output control section 330 alerts to the user 30 by outputting a voice/sound information from the speaker of the input/output section 250. The input/output control section 330 may alert to the user 30 by means of lighting or flashing the lighting apparatus of the input/output section 250. The input/output control section 330 may alert to the user 30 by raising and lowering the vehicle body of the vehicle 20, utilizing the vehicle body raising/lowering apparatus of the input/output section 250.

In this embodiment, the drive control section 340 controls the drive section 220. The drive control section 340 controls the drive section 220 to thereby control at least one of the velocity, the acceleration and the traveling direction of the vehicle 20. In one embodiment, the drive control section 340 may receive a route information indicating the determined driving route from the route search section 350 to thereby control the drive section 220 pursuant to said route information. In another embodiment, the drive control section 340 may receive a control instruction from the assistance server 120 to thereby control the drive section 220 pursuant to said control instruction.

In still another embodiment, the drive control section 340 may control the drive section 220 by utilizing the automatic operation function. At this time, the drive control section 340 may utilize the output information of an external sensor and an internal sensor acquired from the input/output control section 330 to thereby control the drive section 220. For example, the drive control section 340 may receive an information indicating the distance between the vehicle 20 and the user 30 (this is called “user distance” in some cases) from the input/output control section 330 to thereby control the drive section 220 pursuant to said information.

In this embodiment, the route search section 350 determines the driving route for the vehicle 20. The route search section 350 may cooperate with the assistance server 120 to thereby determine the driving route for the vehicle 20. For example, the route search section 350 receives a command for determining the driving route from the input/output control section 330. Moreover, the route search section 350 receives an information indicating the start point S and the destination G of the user 30 from the input/output control section 330.

In this embodiment, the route search section 350 sends the information for requesting the assistance server 120 to search the driving route (this is called “search request” in some cases), to the assistance server 120. The route search section 350 may send, together with the search request, or associated with the search request, the followings to the assistance server 120: (i) the identification information of the user 30, and (ii) the information indicating the start point S and the destination G of the user 30.

In this embodiment, the route search section 350 acquires an information indicating at least a part of the driving route of the vehicle 20 (this is called “route information” in some cases) as the response to the search request. In one embodiment, the route information includes an information indicating the getting-off point D. In this case, the route search section 350 may determine a driving route for the vehicle 20, based on the positions of the start point S and the getting-off point D. Further, the route search section 350 may determine the moving route for the user 30, based on the positions of the getting-off point D and the destination G.

In another embodiment, the route information may include a driving route for the vehicle 20. The driving route for the vehicle 20 includes the information indicating the pathway between the start point S and the getting-off point D. In this case, the route search section 350 may determine the moving route for the user 30, based on the positions of the getting-off point D and the destination G. In still another embodiment, the route information may include the driving route for the vehicle 20 and the moving route for the user 30. The moving route for the user 30 includes the information indicating the pathway between the getting-off point D and the destination G.

The route search section 350 may output the information indicating the driving route for the vehicle 20 to the drive control section 340. The route search section 350 may send the moving route for the user 30 to the communication terminal 32.

In this embodiment, the user information acquisition section 360 acquires various kinds of information related to the user 30. As the various kinds of information related to the user 30, a physical information, a vital information and the like are exemplified. The user information acquisition section 360 may send various kinds of information related to the user 30 to the assistance server 120.

In one embodiment, the user information acquisition section 360 acquires various kinds of information related to the user 30, from the user 30, via the input/output section 250. In another embodiment, the user information acquisition section 360 acquires various kinds of information related to the user 30, from the communication terminal 32, via the communication section 240.

The route search section 350 may be an example of the sending section. The user information acquisition section 360 may be an example of the physical information acquisition section and the vital information acquisition section.

Note that, in this embodiment, detail of the navigation system 100 is explained, taking a case where the route search section 350 sends a search request to the assistance server 120 so that the assistance server 120 determines the getting-off point D, as an example. However, the navigation system 100 is not limited to this embodiment. In another embodiment, the route search section 350 may determine the getting-off point D, cooperating with the assistance server 120, or alone by itself.

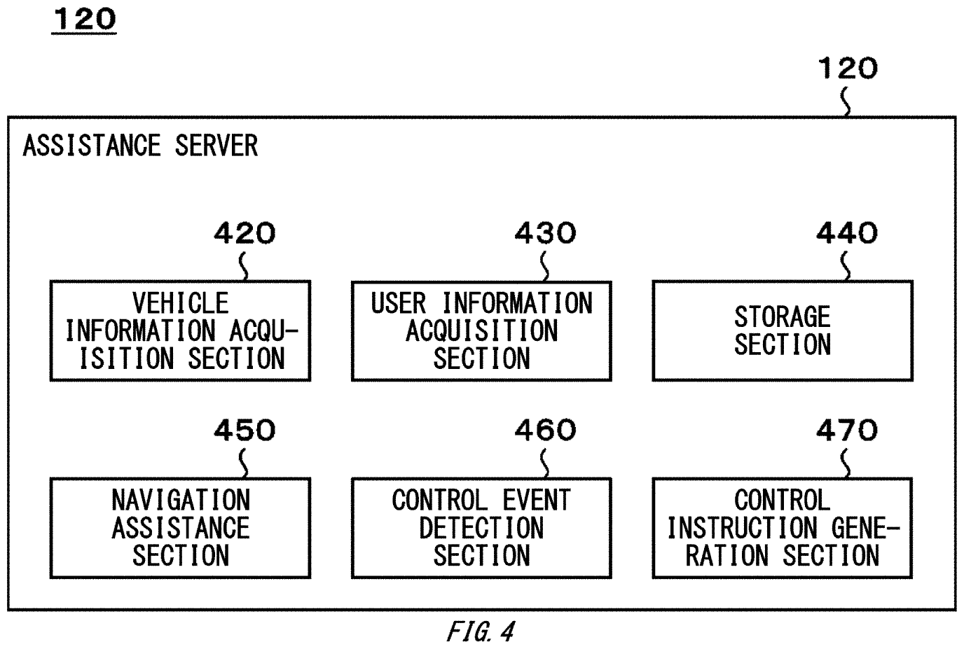

FIG. 4 schematically shows an example of an interior configuration of an assistance server 120. In this embodiment, the assistance server 120 includes a vehicle information acquisition section 420, a user information acquisition section 430, a storage section 440, a navigation assistance section 450, a control event detection section 460, and a control instruction generation section 470.

In this embodiment, the vehicle information acquisition section 420 acquires various kinds of information related to the vehicle 20. For example, the vehicle information acquisition section 420 acquires the information indicating the current position or the moving history of the vehicle 20. The vehicle information acquisition section 420 may acquire the information indicating the state or action status of the vehicle 20. As the state or the action status of the vehicle 20, activating the vehicle 20, stopping the vehicle 20, opening/closing the door of the vehicle 20, getting-on of the user 30, getting-off of the user 30, a velocity of the vehicle 20, an acceleration of the vehicle 20, a traveling direction of the vehicle 20, an energy residual amount of the vehicle 20 and the like are exemplified.

In this embodiment, the user information acquisition section 430 acquires various kinds of information related to the user 30. The user information acquisition section 430 may store various kinds of information related to the user 30 in the storage section 440.

For example, the user information acquisition section 430 acquires the physical information of the user 30. The user information acquisition section 430 may acquire the vital information of the user 30. The user information acquisition section 430 may acquire an information indicating the current position or the moving history of the user 30. The user information acquisition section 430 may acquire the information indicating the current position or the moving history of the communication terminal 32. The user information acquisition section 430 may utilize the current position or the moving history of the communication terminal 32 as the current position or the moving history of the user 30. The user information acquisition section 430 may acquire an activity history information of the user 30 in the facility 40.

In one embodiment, the user information acquisition section 430 may acquire various kinds of information related to the user 30 from the user information acquisition section 360. In another embodiment, the user information acquisition section 430 may acquire various kinds of information related the user 30 from the communication terminal 32.

In still another embodiment, the user information acquisition section 430 may acquire various kinds of information related to the user 30 from another information processing apparatus via the communication network 10. Another information processing apparatus may be a measuring machine for measuring the physical state of the user 30. As the measuring machine, a body height meter, a body weight meter, a heart rate meter, a blood pressure meter and the like are exemplified. Another information processing apparatus may be a personal computer, a server and the like for storing the physical information of the user 30. Another information processing apparatus may be a personal computer, a server and the like for storing the activity history information of the user 30.

In this embodiment, the storage section 440 stores various kinds of information to be utilized in the information processing at the assistance server 120. The storage section 440 stores various kinds of information generated by the information processing at the assistance server 120. Detail of the storage section 440 will be described later.

In this embodiment, the navigation assistance section 450 assists the determination of a driving route for the vehicle 20. The navigation assistance section 450 may determine at least a part of the driving route for the vehicle 20. The navigation assistance section 450 may send the information indicating at least a part of the driving route for the vehicle 20 to the route search section 350.

The navigation assistance section 450 may assist the determination of the moving route for the user 30. The navigation assistance section 450 may determine at least a part of the moving route for the user 30. The navigation assistance section 450 may send the information indicating at least a part of the moving route for the user 30 to the route search section 350. The navigation assistance section 450 may send the information indicating at least a part of the moving route for the user 30 to the communication terminal 32.

For example, the navigation assistance section 450 determines the candidate point for the getting-off point D, based on (i) the position of the destination G of the user 30, and (ii) the physical feature of the user 30. The navigation assistance section 450 may determine one of the one or more candidate points as the getting-off point D. The getting-off point D may be a part of the driving route for the vehicle 20. The getting-off point D may be a part of the moving route of the user 30. Detail of the navigation assistance section 450 will be described later.

In this embodiment, the control event detection section 460 detects an occurrence of an event of the predefined category. If an occurrence of an event of the predefined category is detected, the control event detection section 460 outputs, for example, an information indicating the category of the detected event to the control instruction generation section 470. Detail of the control event detection section 460 will be described later.

In this embodiment, the control instruction generation section 470 acquires an information indicating the category of the event detected by the control event detection section 460. The control instruction generation section 470 controls the action of the vehicle 20 in accordance with the category of the above-described event. Concretely saying, the control instruction generation section 470 generates an instruction for controlling the action of the vehicle 20 (this is called “control instruction” in some cases) in accordance with the category of the above-described event, to accordingly send said control instruction to the drive control section 340. Detail of the control instruction generation section 470 will be described later.

The vehicle information acquisition section 420 may be an example of the getting-off detection section. The user information acquisition section 430 may be an example of the physical information acquisition section, the vital information acquisition section and the user position detection section. The storage section 440 may be an example of the stop location information storage section. The navigation assistance section 450 may be an example of the first destination information acquisition section, the physical information acquisition section, the candidate point determination section, the selection information acquisition section, the second destination determination section, and the sending section. The control event detection section 460 may be an example of the getting-off detection section, the judgment section, the vital information acquisition section, the abnormality detection section, and the arrival detection section. The control instruction generation section 470 may be an example of the alert determination section, the emergency movement determination section, the notification determination section, and the wait determination section.

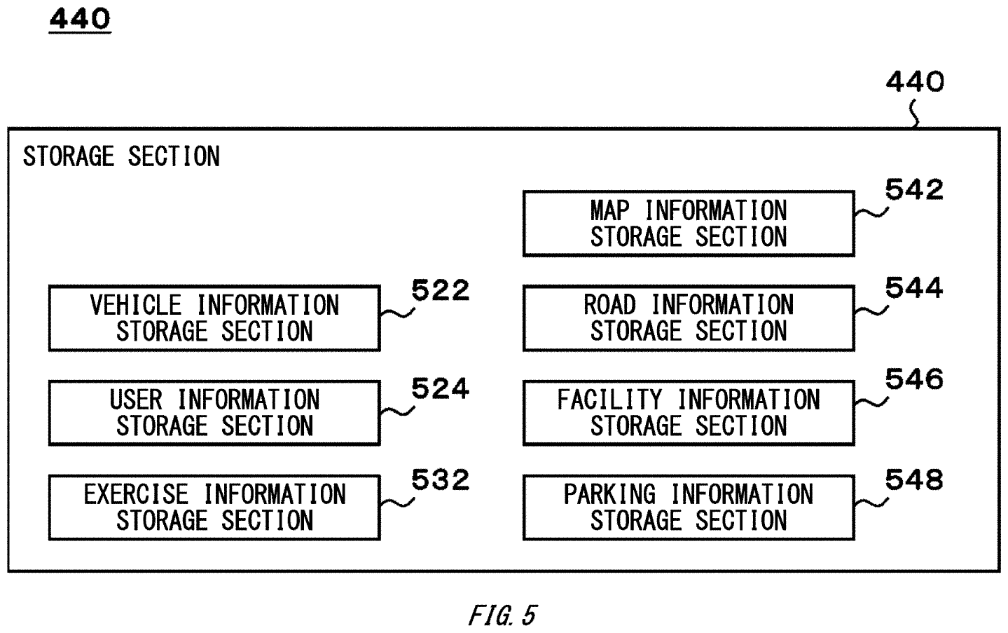

FIG. 5 schematically shows an example of an interior configuration of a storage section 440. In this embodiment, the storage section 440 includes a vehicle information storage section 522, a user information storage section 524, an exercise information storage section 532, a map information storage section 542, a road information storage section 544, a facility information storage section 546 and a parking information storage section 548.

In this embodiment, the vehicle information storage section 522 stores the information acquired by the vehicle information acquisition section 420. In this embodiment, the user information storage section 524 stores the information acquired by the user information acquisition section 430.

In this embodiment, the exercise information storage section 532 stores the information related to the exercise. For example, the exercise information storage section 532 associates (i) the information indicating the category of the exercise and (ii) the information indicating the energy equivalent amount to be consumed by said exercise during a unit time, to accordingly store them. The exercise information storage section 532 may associate (i) the information indicating the intensity of the exercise, and (ii) the information indicating the energy equivalent amount to be consumed by the exercise at said intensity during a unit time, to accordingly store them.

In this embodiment, the map information storage section 542 stores the map information. In this embodiment, the road information storage section 544 stores various kinds of information related to the state of the road. As the state of the road, a presence/absence of road pavement, a category and/or level of the road pavement, a deterioration level of the road, an inclination of the road, an altitude profile of the road and the like are exemplified.

In this embodiment, the facility information storage section 546 stores various kinds of information related to each of one or more facilities 40. As the information related to the facility 40, information indicating (i) an identification information of the facility 40, (ii) a category of the facility 40, (iii) a category of the service provided in the facility 40, (iv) a calorie amount or a nutrient ingredient in the meal offered in the facility 40, (v) an energy equivalent amount to be consumed by the exercise or action of the user 30 in the facility 40, and the like, are exemplified.

The information related to the facility 40 may include the information indicating a statistic amount related to the calorie amount or nutrient ingredient amount of a meal provided in the facility 40. The information related to the facility 40 may include the information indicating the statistic amount related to the energy equivalent amount to be consumed by the exercise or action of the user 30 in the facility 40. As the statistic amount, a maximum value, a minimum value, an average value, a median value and the like are exemplified. The statistic amount may be a statistic amount related to the meals or services of all the categories to be provided in the facility 40, or may be a statistic amount related to the meals or services utilized by multiple users 30.

In this embodiment, the parking information storage section 548 stores the information related to the parkings where the vehicle 20 can be parked or stopped. As the information related to the parkings, (i) an identification information of the parking, (ii) an information indicating the position of the parking, (iii) an information indicating the vacancy status of the parking, (iv) an information indicating the usage fee of the parking, and the like, are exemplified. The parking information storage section 548 preferably stores at least the following information associated to each other: (i) an identification information of the parking, and (ii) an information indicating the position of the parking.

The parking information storage section 548 may be an example of the stop location information storage section. The parking may be an example of a stop location.

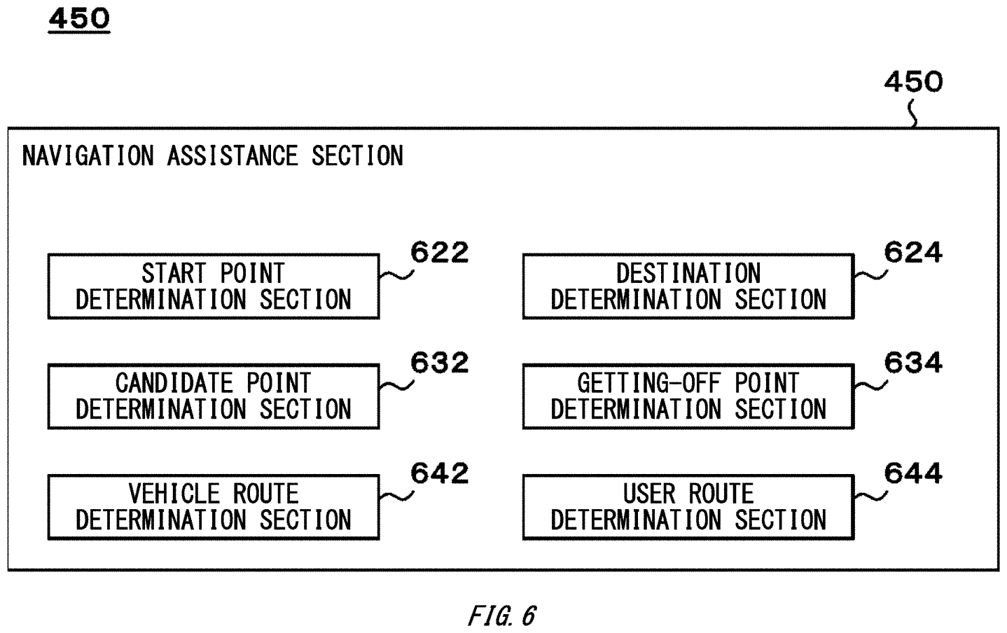

FIG. 6 schematically shows an example of an interior configuration of a navigation assistance section 450. In this embodiment, the navigation assistance section 450 includes a start point determination section 622, a destination determination section 624, a candidate point determination section 632, a getting-off point determination section 634, a vehicle route determination section 642 and a user route determination section 644.

In this embodiment, the start point determination section 622 acquires the information indicating the start point S of the user 30. For example, the start point determination section 622 acquires, from the route search section 350, (i) the identification information of the user 30, and (ii) the information indicating the start point S and the destination G of the user 30. The start point determination section 622 may determine the start point S indicated by the above-described information as the start point of the vehicle 20.

In this embodiment, the destination determination section 624 acquires the information indicating the destination G of the user 30. For example, the destination determination section 624 acquires, from the route search section 350, (i) the identification information of the user 30, and (ii) the information indicating the start point S and the destination G of the user 30. The destination determination section 624 may determine the destination G indicated by the above-described information as the destination G of the user 30.

In this embodiment, a candidate point for the getting-off point D is determined. The candidate point determination section 632 may determine a single point as the candidate point for the getting-off point D, or may determine multiple points as the candidate points for the getting-off point D. The candidate point determination section 632 may output the information indicating the candidate point for the getting-off point D to the getting-off point determination section 634.

Concretely saying, for example, the candidate point determination section 632 determines the candidate point for the getting-off point D, pursuant to the procedure as described below. Firstly, the candidate point determination section 632 refers to the user information storage section 524 to acquire the physical information of the user 30, taking the identification information of the user 30 as the key. Next, the candidate point determination section 632 acquires the information indicating the destination G of the user 30 from the destination determination section 624. The candidate point determination section 632 determines the candidate point for the getting-off point D, based on (i) the position of the destination G of the user 30, and (ii) the physical feature of the user 30.

More concretely saying, firstly, the candidate point determination section 632 determines the target value of the energy equivalent amount to be consumed if the user 30 moves from the getting-off point D to the destination G by means of physical activity of the user 30 without utilizing the vehicle 20. Next, the candidate point determination section 632 determines the candidate point for getting-off point D, based on, for example, (i) the position of the destination G of the user 30, and (ii) the target value of the above-described energy equivalent amount.

The determination method of the above-described target value is not particularly limited. In one embodiment, the candidate point determination section 632 determines the above-described target value based on the input related to the above-described target value by the user 30. In another embodiment, the candidate point determination section 632 determines the above-described target value based on the physical feature of the user 30.

For example, firstly, the candidate point determination section 632 determines the target, which is to consume 100 kcal by means of moving by walking from the getting-off point D to the destination G. Next, the candidate point determination section 632 calculates the distance necessary for consuming energy of 100 kcal by walking, taking into account, for example, the gender, the age and the body weight of the user 30. Next, the candidate point determination section 632 extracts a position appropriate for the user 30 to get off the vehicle 20, from among the positions or the regions which are distant from the calculated distance, as the candidate point for the getting-off point D. Detail of the candidate point determination section 632 will be described later.

In this embodiment, the getting-off point determination section 634 determines the getting-off point D. Concretely saying, the getting-off point determination section 634 determines the getting-off point D, for example, pursuant to the procedure as described below. Firstly, the getting-off point determination section 634 acquires the information indicating the candidate point of the getting-off point D from the candidate point determination section 632. The getting-off point determination section 634 generates a screen for offering the candidate point for the getting-off point D to the user 30, to accordingly send the data of said screen to the route search section 350. The route search section 350 forwards the data of the screen to the input/output control section 330.

The input/output control section 330 controls the input/output section 250, to accordingly display, for example, the above-described screen on the display apparatus of the input/output section 250. The user 30 selects one of the one or more candidate points displayed on the screen to input the information indicating said selected result to the input/output section 250. The input/output control section 330 acquires the information indicating the candidate point selected by the user 30 from the input/output section 250, to send said information to the getting-off point determination section 634.

The getting-off point determination section 634 acquires the information indicating the candidate point selected by the user 30. The getting-off point determination section 634 determines the candidate point selected by the user 30 as the getting-off point D. The getting-off point determination section 634 outputs the information indicating the getting-off point D to the vehicle route determination section 642 and the user route determination section 644.

In this embodiment, the vehicle route determination section 642 determines at least a part of the driving route of the vehicle 20. For example, the vehicle route determination section 642 determines to set the start point of the vehicle 20 as the start point S and the destination of the vehicle 20 as the getting-off point D. The vehicle route determination section 642 may determine the pathway between the start point S and the getting-off point D. The vehicle route determination section 642 may send the information indicating at least a part of the driving route for the vehicle 20 to the route search section 350.

Note that, the vehicle route determination section 642 may determine, after starting the start point S and arriving at the getting-off point D, the route to move to the vicinity of the destination G following after the user 30, as the driving route for the vehicle 20. The vehicle route determination section 642 may determine, after starting the start point S and arriving at the getting-off point D, and further after moving to the vicinity of the destination G following after the user 30, the route to move to the getting-off point D, as the driving route for the vehicle 20.

By this, the vehicle 20 is positioned behind the traveling direction of the user 30, during the period when the user 30 moves from the getting-off point D to the destination G. By this, the user 30 can again get on the vehicle 20 at any timing. At this time, the vehicle 20 may move following after the user 30 so that the distance between the vehicle 20 and the user 30 should be within a predefined range.

In this embodiment, the user route determination section 644 determines at least a part of the moving route of the user 30. For example, the user route determination section 644 determines to set the start point of the user 30 as the getting-off point D, and the destination of the user 30 as the destination G. The user route determination section 644 may determine the pathway between the getting-off point D and the destination G. The user route determination section 644 may send the information indicating at least a part of the moving route for the user 30 to the route search section 350. The user route determination section 644 may send the information indicating at least a part of the moving route for the user 30 to the communication terminal 32.

The destination determination section 624 may be an example of the first destination information acquisition section. The candidate point determination section 632 may be an example of the candidate point determination section. The getting-off point determination section 634 may be an example of the selection information acquisition section and the second destination determination section. The vehicle route determination section 642 may be an example of the following movement determination section. The user route determination section 644 may be an example of the sending section. The information indicating the destination G of the user 30 may be an example of the first destination information. The information indicating the candidate point selected by the user 30 may be an example of the user selection information.

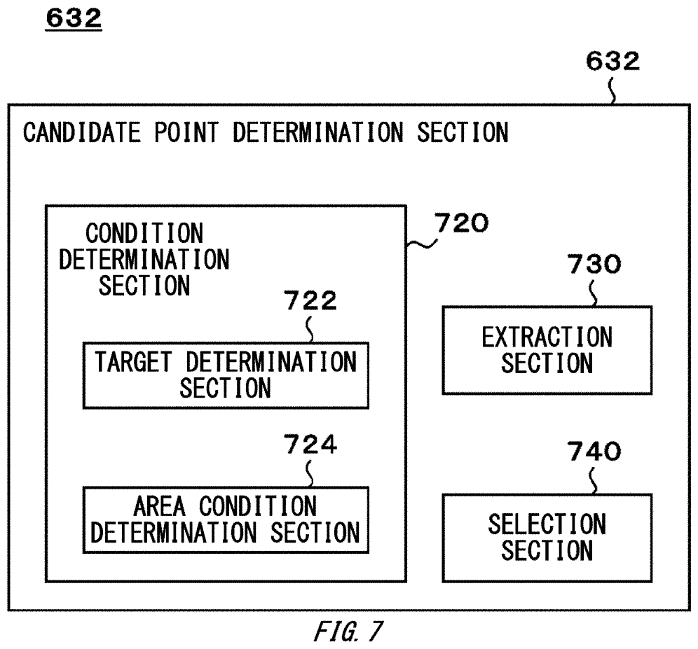

FIG. 7 schematically shows an example of an interior configuration of a candidate point determination section 632. In this embodiment, the candidate point determination section 632 includes a condition determination section 720, an extraction section 730 and the selection section 740. In this embodiment, the condition determination section 720 has a target determination section 722 and an area condition determination section 724.

In this embodiment, the condition determination section 720 determines the condition to be the object of extraction for the extraction section 730. For example, the condition determination section 720 determines the condition for extracting a parking which can be a candidate point for the getting-off point D from among the parkings existing in the periphery of the destination G.

In this embodiment, the target determination section 722 determines the target value of the energy equivalent amount to be consumed if the user 30 moves from the getting-off point D to the destination G by the physical activity of the user 30 without utilizing the vehicle 20. The target determination section 722 may determine the category of the physical activity. The target determination section 722 may output the information indicating the above-described target value to the area condition determination section 724. The target determination section 722 may output the information indicating the category of the physical activity to the area condition determination section 724.

In one embodiment, the target determination section 722 determines the above-described target value, for example, based on the input by the user 30. As described above, the user 30 inputs, for example, an energy equivalent amount, which he/she desires to consume by means of physical activity during the period until he/she arrives to the destination G, to the communication terminal 32 or the input/output section 250 of the vehicle control system 110. The user 30 may input an energy equivalent amount, which he/she desires to consume by means of physical activity during the period when he/she reciprocates between the start point S and the destination G.

The target determination section 722 acquires the information inputted by the user 30, from the communication terminal 32 or the vehicle control system 110. The target determination section 722 may determine the above-described target value, based on the information inputted by the user 30. For example, the target determination section 722 determines the energy equivalent amount, which the user 30 desires to consume by means of physical activity during the period until he/she arrives to the destination G, as the above-described target value. The target determination section 722 may determine the half of the energy equivalent amount, which the user 30 desires to consume by means of physical activity during the period when he/she reciprocates between the start point S and the destination G, as the above-described target value.

As described above, the user 30 may input a target related to a diet or a training to the input/output section 250 of the communication terminal 32 or the vehicle control system 110. The above-described target may include an information indicating an ideal value related to at least one of a body weight, a muscle mass and a body fat percentage. The above-described target may include the information indicating the ideal value related to at least one of an information indicating a time limit of a diet or a training, and a body weight, a muscle mass and a body fat percentage at the time of said time limit.

The target determination section 722 may determine the target value of the above-described energy equivalent amount, based on the target related to the above-described diet or training. For example, the target determination section 722 determines the target value of the energy equivalent amount to be consumed when the user 30 moves from the getting-off point D to the destination G, based on the remaining time to the limit and the current value of at least one of the body weight, the muscle mass and the body fat percentage of the user 30.

When the navigation system 100 manages the activity history of the user 30, the target determination section 722 may set the target value of the energy equivalent amount to be consumed by the exercise of the user 30 by every unit period. The target determination section 722 may determine the target value of the energy equivalent amount to be consumed when the user 30 moves from the getting-off point D to the destination G, based on the difference between the energy equivalent amount already consumed by the exercise of the user 30 in a given unit period and the target value in said given unit period. The target determination section 722 may determine the target value of the energy equivalent amount to be consumed when the user 30 moves from the getting-off point D to the destination G, based on the energy equivalent amount already consumed by the exercise of the user 30 during a given unit period, the energy equivalent amount ingested by the user 30 by means of eating a meal during said given unit period, and the target value in said given unit period.

The length of the unit period is preferably one day or more, though it is not particularly limited. As the unit period, one day, two days, three days, one week, two weeks, one month, two months, three months, six months, one year and the like are exemplified. The energy consumption amount resulting from an exercise may be the one, from the energy consumption amount of the user 30, from which the energy consumption amount resulting from non-exercise-based physical activities such as daily living activities, the energy consumption amount resulting from the basal metabolic rate, and the energy consumption amount resulting from the meal-induced thermogenesis, are excluded.

In another embodiment, the target determination section 722 determines the above-described target value, for example, based on the physical feature of the user 30 indicated by the physical information. The target determination section 722 may refer to the storage section 440 to acquire the information related to the physical feature of the user 30 acquired by the user information acquisition section 430. At least a part of the information indicating the physical feature of the user 30 may be the information inputted by the user 30 to the input/output section 250 of the communication terminal 32 or the vehicle control system 110. At least a part of the information indicating the physical feature of the user 30 may be the information acquired from a measuring machine or the like by the user information acquisition section 430.

For example, the physical information includes an information indicating the current value and the ideal value of at least one of the body weight, the muscle mass and the body fat percentage. In this case, the target determination section 722 may determine the above-described target value, based on the difference between the above-described current value and ideal value. The current value may be the value acquired by means of the most recent measurement. The current value may be the last value inputted by the user 30.

The ideal values of the body weight, the muscle mass or the body fat percentage may be the value inputted by the user 30, or may be the value inputted as a default. The ideal values of the body weight, the muscle mass or the body fat percentage may be determined using a table, a function or a model which takes at least one of the citizenship, the age, the gender and the body height as the explanatory variable, and also takes the ideal value of the body weight, the muscle mass or the body fat percentage as the objective variables.

The more the number of categories of explanatory variables inputted to the above-described function or model is, the better the precision of the ideal values improve. However, input of the value of the explanatory valuables of all the categories is not required. For example, if an information like “thirties” and “man” is inputted to the model, said model outputs values of the body weight, the muscle mass, the body fat percentage and the like of a “man in his thirties” having an average body mass.

For example, the target determination section 722 refers to the storage section 440 to acquire the information indicating the current value of the body weight of the user 30. The target determination section 722 refers to the storage section 440 to acquire the information indicating the age, the gender and the body height of the user 30. The target determination section 722 inputs, for example, the information indicating the age, the gender and the body height of the user 30, to accordingly acquire the body weight value outputted by said model as the ideal body weight value for the user 30. The target determination section 722 determines the target value of the above-described energy equivalent amount, based on the difference between the current value and the ideal value of the body weight of the user 30. The target determination section 722 may determine the category of the physical activity, based on the difference between the current value and the ideal value of the body weight of the user 30. For example, if the current value of the body weight of the user 30 is greater than the ideal value, the target determination section 722 determines said target value so that the greater the absolute value of the difference between the current value and the ideal value of the body weight of the user 30 is, the greater said target value be. The target determination section 722 selects a walking or a light jogging as the category of physical activity.

In still another embodiment, the target determination section 722 determines the above-described target value, based on the category of the facility 40 or the category of the service. For example, the target determination section 722 refers to the facility information storage section 546 to acquire the information indicating the category of the facility 40 or the category of the service provided in the facility 40 (this is called “category information” in some cases), taking the identification information of the facility 40 positioned at the destination G as the key. The target determination section 722 may determine the above-described target value, based on the category of the facility 40 or the category of the service indicated by the category information. The target determination section 722 may determine the above-described target value, based on (i) the physical feature of the user 30 indicated by the physical information, and (ii) the category of the facility 40 or the category of the service indicated by the category information.

For example, if the facility 40 is a restaurant, the target determination section 722 determines said target value so that the greater the calorie amount of a meal provided in said facility is, the greater the above-described target value be. The calorie amount of the meal provided in the facility 40 may be determined based on a statistical data of the meals provided in the facility 40.

For example, if the facility 40 is a training gym, the target determination section 722 determines said target value so that the greater the intensity of the exercise at said facility is, the smaller the above-described target value be. The intensity of the exercise in the facility 40 may be determined based on a statistical data of the exercises performed in the facility 40.

In still another embodiment, the target determination section 722 may randomly determine the above-described target value. The target determination section 722 may determine the above-described target value, so that the average value of the target values which have been randomly determined in multiple attempts should be the value determined pursuant to the above-described procedure, based on the physical feature of the user 30 indicated by the physical information.

In this embodiment, the area condition determination section 724 determines the condition related to the position of a parking which can be the candidate point for the getting-off point D. The area condition determination section 724 may determine the above condition, based on the physical feature of the user 30. The area condition determination section 724 may determine the above-described condition, based on the target value of the energy equivalent amount to be consumed if the user 30 moves from the getting-off point D to the destination G, and the physical feature of the user 30. The above-described condition, which may be the condition related to the distance between the candidate point for the getting-off point D and the destination G, may be the condition related to the distance of the moving pathway from the candidate point for the getting-off point D to the destination G. The area condition determination section 724 outputs the information indicating the condition related to the position of the parking, which can be the candidate point for the getting-off point D, to the extraction section 730.

For example, the area condition determination section 724 acquires the information indicating the target value of the energy equivalent amount to be consumed if the user 30 moves from the getting-off point D to the destination G, from the target determination section 722. The area condition determination section 724 refers to the user information storage section 524 to thereby acquire the information indicating at least one of the current value of the body weight, the age and the gender of the user 30. The area condition determination section 724 determines the condition related to the distance between the candidate point for the getting-off point D and the destination G, based on (i) the above-described target value, and (ii) at least one of the current value of the body weight, the age and the gender of the user 30. The area condition determination section 724 may determine at least one of the upper limit and the lower limit of the above-described distance.

For example, an information specifying that the calorie consumption amount by means of walking is 100 kcal is inputted as the target value of the energy equivalent amount, and an information specifying that the user 30 is a man in his thirties as the physical feature of the user 30, to the area condition determination section 724. In this case, the area condition determination section 724 outputs the condition that the distance to the destination G is 2 km, as the condition related to the position of the parking which can be the candidate point for the getting-off point D. The area condition determination section 724 may output the condition that the distance to the destination G is between 1.8 km and 2.2 km, as the condition related to the position of the parking which can be the candidate point for the getting-off point D.

The condition related to the distance between the candidate point for the getting-off point D and the destination G may be an example of a condition related to the position of the parking which may be the candidate point for the getting-off point D. The area condition determination section 724 may determine the upper limit and the lower limit of the above-described distance. By this, the area having certain geographical area is identified.

In another embodiment, the area condition determination section 724 may acquire the information indicating the moving mode when the user 30 moves from the getting-off point D to the destination G (this is called “moving mode information” in some cases). The area condition determination section 724 may acquire, for example, the moving mode information from the user 30 via the input/output section 250. As the moving mode, a walking, a rapid walking, a jogging, a running, a cycling and the like are exemplified. The area condition determination section 724 may determine the condition related to the distance between the candidate point for the getting-off point D and the destination G, based on (i) the above-described target value, (ii) the current body weight of the user 30, and (iii) the moving mode indicated by the moving mode information. The area condition determination section 724 may determine at least one of the upper limit and the lower limit of the above-described distance.

In another embodiment, the area condition determination section 724 may acquire the information indicating the time at which the user 30 desires to arrive at the destination G (this is called “desired time information” in some cases). The area condition determination section 724 may acquire the desired time information from the user 30 via the input/output section 250. The area condition determination section 724 may determine the condition related to the distance between the candidate point for the getting-off point D and the destination G, based on (i) the above-described target value, (ii) at least one of the current body weight, the age and the gender of the user 30, and (iii) the time indicated by the desired time information. The area condition determination section 724 may determine at least one of the upper limit and the lower limit of the above-described distance.

For example, the area condition determination section 724 determines the lower limit value of the distance between the candidate point for the getting-off point D and the destination G, based on (i) the above-described target value, and (ii) at least one of the current value of the body weight, the age and the gender of the user 30. The area condition determination section 724 determines the distance with which the user 30 can arrive at the destination G by the time indicated by the desired time information as the upper limit value of the distance between the candidate point for the getting-off point D and the destination G.

In another embodiment, the area condition determination section 724 refers to the road information storage section 544 to acquire the information indicating the road surface status or the altitude profile of the roads in the periphery of the destination G (this is called “road information” in some cases). The area condition determination section 724 may determine the condition related to the distance between the candidate point for the getting-off point D and the destination G, based on (i) the above-described target value, (ii) at least one of the current body weight, the age and the gender of the user 30, and (iii) at least one of the road surface status and the altitude profile of the roads indicated by the road information. The area condition determination section 724 may determine at least one of the upper limit and the lower limit of the above-described distance.

For example, the area condition determination section 724 determines the above-described condition so that the more the unpaved roads exist in the periphery of the destination G, the shorter the distance between the candidate point for the getting-off point D and the destination G be. The area condition determination section 724 may determine the above-described condition so that the greater the inclination of the roads in the periphery of the destination G is, the shorter the distance between the candidate point for the getting-off point D and the destination G be.

In this embodiment, the extraction section 730 extracts a parking that meets the condition determined by the area condition determination section 724 from among the parkings existing in the periphery of the destination G. Concretely saying, for example, the extraction section 730 firstly acquires the information indicating the condition related to the position of a parking which can be the candidate point for the getting-off point D from the area condition determination section 724. Next, the extraction section 730 refers to the parking information storage section 548 to extract a parking that meets the condition determined by the area condition determination section 724 from among the parkings existing in the periphery of the destination G. The extraction section 730 may output the identification information of the extracted parking to the selection section 740.

In this embodiment, the selection section 740 acquires the identification information of the extracted parking from the extraction section 730. The selection section 740 selects at least one of the parkings extracted by the extraction section 730 as the candidate point for the getting-off point D. In one embodiment, the selection section 740 selects all the parkings extracted by the extraction section 730 as the candidate point for the getting-off point D.

In another embodiment, the selection section 740 firstly acquires, for each of the parkings extracted by the extraction section 730, the information indicating at least one of the distance of the pathway from said parking to the destination G, the road surface status of said pathway, and the altitude profile of said pathway. For example, the selection section 740 refers to the map information storage section 542 to acquire the information indicating the distance of the pathway from each parking to the destination G. The selection section 740 may refer to the road information storage section 544 to acquire the information indicating at least one of the road surface status of the pathway from each parking to the destination G and the altitude profile of said pathway.

Next, the selection section 740 estimates the energy equivalent amount to be consumed if the user 30 moves from each parking to the destination G by means of the physical activity of the user 30, based on at least one of the distance of the above-described pathway, the road surface status of said pathway and the altitude profile of said pathway. The selection section 740 may refer to the information stored in the exercise information storage section 532 to estimate the above-described energy equivalent amount.

Next, the selection section 740 selects at least one of the one or more parking extracted by the extraction section 730 as the candidate point for the getting-off point D, based on the estimated value of the above-described energy equivalent amount. For example, the selection section 740 selects at least one of the one or more parking extracted by the extraction section 730 as the candidate point for the getting-off point D, based on the difference between the above-described estimated value and the target value determined by the target determination section 722.

More concretely saying, if the absolute value of the difference between the above-described estimated value and the above-described target value is within a predefined range for given parking, the selection section 740 may select said parking as the candidate point for the getting-off point D. The selection section 740 may select a predefined number of parkings as the candidate point for the getting-off point D, from the ones which have smaller absolute value of the difference between the above-described estimated value and the above-described target value.

The selection section 740 may select at least one of the one or more parking extracted by the extraction section 730 as the candidate point for the getting-off point D, based on (i) the information indicating the estimated value of the above-described energy equivalent amount, and (ii) at least one of the physical information, the category information, the moving mode information, the desired time information and the road information. For example, if an information indicating that the user 30 has an injury in his/her knee is included in the physical information, the selection section 740 evaluates, for each parking, the altitude difference of the pathways between said parking and the destination G. If the altitude difference satisfies the predefined criterion, the selection section 740 may select said parking as the candidate point for the getting-off point D.

The target determination section 722 may be an example of the physical information acquisition section or the category information acquisition section. The area condition determination section 724 may be an example of the distance condition determination section, the mode information acquisition section, the time information acquisition section and the road information acquisition section. The extraction section 730 may be an example of the extraction section. The selection section 740 may be an example of the selection section.

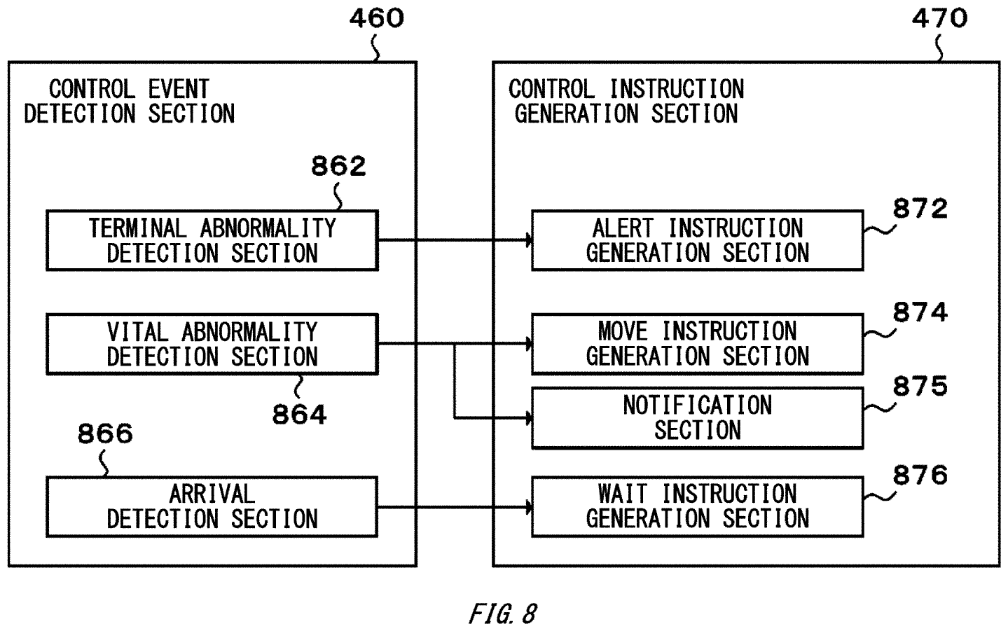

FIG. 8 schematically shows an example of an interior configuration of a control event detection section 460 and a control instruction generation section 470. In this embodiment, the control event detection section 460 includes a terminal abnormality detection section 862, the vital abnormality detection section 864 and the arrival detection section 866. In this embodiment, the control instruction generation section 470 includes an alert instruction generation section 872, a move instruction generation section 874, a notification section 875 and a wait instruction generation section 876.

In this embodiment, the terminal abnormality detection section 862 detects that an abnormality or a sign of an abnormality has occurred to the communication terminal 32. For example, if the user 30 gets off the vehicle 20 with the communication terminal 32 left in the vehicle 20 at the getting-off point D, the terminal abnormality detection section 862 detects that an abnormality or a sign of an abnormality has occurred to the communication terminal 32.

More concretely saying, the terminal abnormality detection section 862 firstly detects that the user 30 has got off the vehicle 20 at the getting-off point D. For example, the terminal abnormality detection section 862 refers to the vehicle information storage section 522 to acquire the information indicating the stop position of the vehicle 20 and the information indicating the open/close status of the doors of the vehicle 20. For example, if it was detected that the vehicle 20 stopped at the getting-off point D and also that a door was opened and closed at the getting-off point D, the terminal abnormality detection section 862 detects that the user 30 got off the vehicle 20 at the getting-off point D.

If it was detected that the user 30 has got off the vehicle 20 at the getting-off point D, the terminal abnormality detection section 862 refers to the user information storage section 524 to monitor the position of the communication terminal 32. The terminal abnormality detection section 862 refers to the vehicle information storage section 522 to monitor the position of the vehicle 20. The terminal abnormality detection section 862 judges whether the distance between the vehicle 20 and the communication terminal 32 is within the predefined range.

The terminal abnormality detection section 862 judges that the user 30 left the communication terminal 32 in the vehicle 20, if (i) the predefined time has passed after detecting that the user 30 got off the vehicle 20 at the getting-off point D, and also (ii) it is judged that the distance between the vehicle 20 and the communication terminal 32 is within the predefined range. In this case, the terminal abnormality detection section 862 outputs the information indicating that an abnormality or a sign of an abnormality has occurred to the communication terminal 32, for example, to the alert instruction generation section 872 of the control instruction generation section 470.

In this embodiment, the vital abnormality detection section 864 detects that an abnormality or a sign of an abnormality has occurred to the vital of the user 30. For example, the vital abnormality detection section 864 detects that an abnormality or a sign of an abnormality has occurred to the vital of the user 30, during the period when the user 30 moves from the getting-off point D to the destination G.

More concretely saying, firstly, the vital abnormality detection section 864 detects that the user 30 got off the vehicle 20 at the getting-off point D, based on the above-described procedure. Next, the vital abnormality detection section 864 refers to the user information storage section 524 to acquire the vital information of the user 30, during the period when the user 30 moves from the getting-off point D to the destination G.

Next, the vital abnormality detection section 864 detects a possibility of potential occurrence of an abnormality to the body of the user 30, or a possibility of actual occurrence of an abnormality to the body of the user 30, based on the vital information of the user 30. If the possibility of potential occurrence of an abnormality to the body of the user 30 or the possibility of actual occurrence of an abnormality to the body of the user 30 was detected, the vital abnormality detection section 864 outputs the information indicating that an abnormality or a sign of an abnormality occurred to the vital of the user 30, for example, to the move instruction generation section 874 of the control instruction generation section 470 and the notification section 875.

In this embodiment, the arrival detection section 866 detects that the user 30 arrived at the destination G. As described above, in some cases, the route to move to the vicinity of the destination G following after the user 30 after starting from the start point S and thereafter arriving at the getting-off point D is determined by the vehicle route determination section 642 to be the driving route for the vehicle 20. The arrival detection section 866 may detect that the user 30 has arrived at the destination G in such a case.

More concretely saying, the arrival detection section 866 firstly detects that the user 30 has got off the vehicle 20 at the getting-off point D, based on the above-described procedure. Next, if it is detected that the user 30 has got off the vehicle 20 at the getting-off point D, the arrival detection section 866 refers to the user information storage section 524 to monitor the position of the user 30. The arrival detection section 866 may detect the position of the user 30 by means of detecting the position of the communication terminal 32.

By this, the arrival detection section 866 can detect that the user 30 has arrived at the destination G, based on the detected position of the user 30. If it is detected that the user 30 has arrived at the destination G, the vital abnormality detection section 864 outputs the information indicating that the user 30 has arrived at the destination G, for example, to the wait instruction generation section 876 of the control instruction generation section 470.

In this embodiment, the alert instruction generation section 872 acquires the information indicating that an abnormality or a sign of an abnormality has occurred to the communication terminal 32, from the terminal abnormality detection section 862. The alert instruction generation section 872 determines, after acquiring the above-described information, to issue an alert to the user 30. Moreover, the alert instruction generation section 872 may generate a control instruction for making the vehicle control system 110 of the vehicle 20 execute a procedure to issue an alert to the user 30 (this is called “alert instruction” in some cases). The alert instruction generation section 872 may send the alert instruction to the vehicle control system 110.

The alert instruction may include at least one of the information indicating the content of the alert message and the information indicating the mode of the alert. The content of the alert message may include the information indicating the category of the abnormality. For example, the content of the alert message may include an information suggesting the possibility that the user 30 may have left the communication terminal 32 in the vehicle 20. As the mode of the alert, an alert by means of voice/sound message, an alert by means of the horn, an alert by means of a light, an alert by means of a vehicle body raising/lowering of the vehicle 20 and the like are exemplified.

In this embodiment, the move instruction generation section 874 acquires the information indicating that an abnormality or a sign of an abnormality has occurred to the vital of the user 30, from the vital abnormality detection section 864. The move instruction generation section 874 determines, after acquiring the above-described information, to move the vehicle 20 to the position of the user 30.

Moreover, the move instruction generation section 874 may generate a control instruction for making the vehicle control system 110 of the vehicle 20 execute a procedure to move the vehicle 20 to the position of the user 30 (this is called “emergency move instruction” in some cases). The emergency move instruction may include the information indicating the position of the user 30 at the most recent time before the time when said instruction was generated. The move instruction generation section 874 may send the emergency move instruction to the vehicle control system 110.

In this embodiment, the notification section 875 acquires the information indicating that an abnormality or a sign of an abnormality has occurred to the vital of the user 30, from the vital abnormality detection section 864. The notification section 875 determines, after acquiring the above-described information, to notify to the predefined emergency notification destination. As the emergency notification destination, an emergency life-saving agency, a security agency, an investigation agency, a family member, a relative, a friend, an acquaintance, a workplace and the like are exemplified.

Further, the notification section 875 may notify the information indicating that an abnormality or a sign of an abnormality has occurred to the vital of the user 30 to the above-described emergency notification destination. The notification section 875 may notify the information indicating the position of the user 30 at the most recent time, to the above-described emergency notification destination.

In this embodiment, the wait instruction generation section 876 acquires the information indicating that the user 30 has arrived at the destination G, from the arrival detection section 866. As described above, in some cases, the route to move to the vicinity of the destination G following after the user 30 after starting from the start point S and thereafter arriving at the getting-off point D is determined by the vehicle route determination section 642 to be the driving route for the vehicle 20. In this case, the navigation system 100 is expected to guide the vehicle 20 to an appropriate position, after the user 30 arrives at the destination G.

Then, according to this embodiment, the wait instruction generation section 876 determines, after acquiring the above-described information, to move the vehicle 20 to the wait location for the vehicle 20

In one embodiment, the wait instruction generation section 876 determines to move the vehicle 20 to the getting-off point D. In another embodiment, the wait instruction generation section 876 determines to move the vehicle 20 to the parking in the periphery of the destination G.

Moreover, the wait instruction generation section 876 may generate a control instruction for making the vehicle control system 110 of the vehicle 20 execute a procedure to move the vehicle 20 to the wait location of the vehicle 20 (this is called “wait instruction” in some cases). The wait instruction may include the information indicating the position of the wait location of the vehicle 20. The wait instruction generation section 876 may send the wait instruction to the vehicle control system 110.

The terminal abnormality detection section 862 may be an example of the getting-off detection section and the judgment section. The vital abnormality detection section 864 may be an example of the vital information acquisition section and the abnormality detection section. The arrival detection section 866 may be an example of the user position detection section. The alert instruction generation section 872 may be an example of the alert determination section. The move instruction generation section 874 may be an example of the emergency move determination section. The notification section 875 may be an example of the notification determination section. The wait instruction generation section 876 may be an example of the wait determination section.

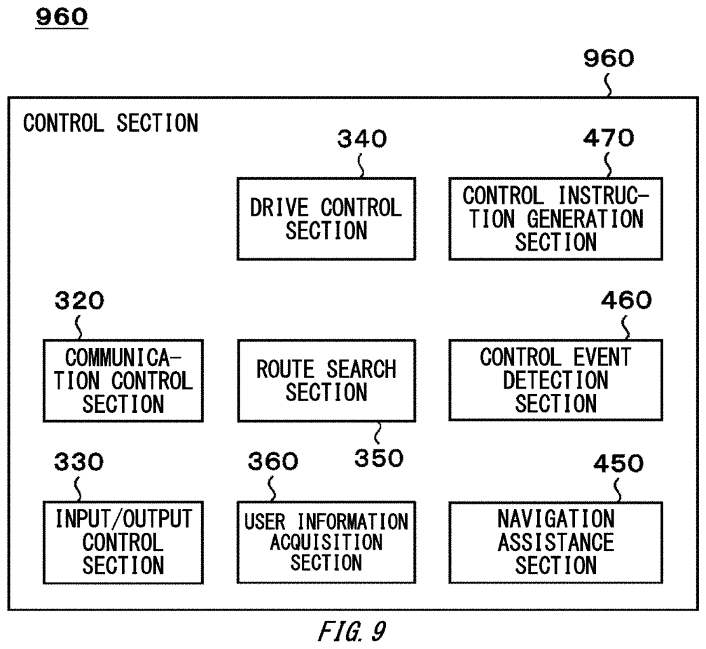

FIG. 9 schematically shows an example of an interior configuration of a control section 960. The control section 960 may be another embodiment of the control section 260 which is explained related to FIG. 2 and FIG. 3.

As described above, in FIG. 2 and FIG. 3, detail of the control section 260 has been explained, taking a case, where the route search section 350 of the control section 260 sends a search request to the assistance server 120 and accordingly the assistance server 120 determines the getting-off point D, as an example. The control section 960 has a part of the function of the assistance server 120, which is the different point from the control section 260. For features other than the above-described difference, the control section 960 may have a similar configuration with that of the control section 260.

In this embodiment, the control section 960 includes a communication control section 320, an input/output control section 330, a drive control section 340, a route search section 350 and a user information acquisition section 360, similarly to the control section 260. The control section 960 further includes a navigation assistance section 450, a control event detection section 460 and a control instruction generation section 470.

By this, according to this embodiment, the vehicle control system 110 determines the candidate point for the getting-off point D, based on (i) the position of the destination G of the user 30, and (ii) the physical feature of the user 30. Moreover, the vehicle control system 110 determines the getting-off point D.

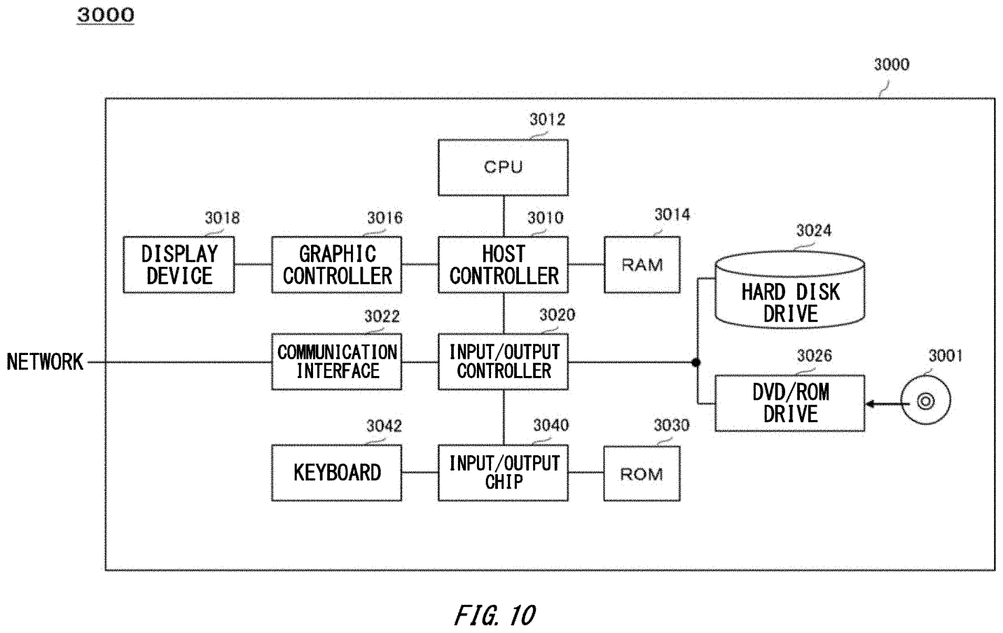

FIG. 10 schematically shows an example of a computer 3000, in which multiple embodiments of the present invention may be entirely or partly realized. The navigation system 100 or a part thereof may be realized by the computer 3000. For example, each of the vehicle control system 110 and the assistance server 120 is realized by the computer 3000.

The program installed in the computer 3000 can make the computer 3000 function as an operation associated with an apparatus according to the embodiment of the present invention or as one or more “section” of said apparatus, or make it execute said operation or said one or more “section”, and/or make the computer 3000 execute a process according to the embodiment of the present invention or the step of said process. Such program may be executed by the CPU 3012, so as to make the computer 3000 execute given operation associated with some or all of the flowcharts and the blocks of the block diagrams described in this specification.

The computer 3000 according to this embodiment includes the CPU 3012, the RAM 3014, the graphic controller 3016 and the display device 3018, and they are connected to each other by the host controller 3010. The computer 3000 further includes the communication interface 3022, the hard disk drive 3024, the DVD-ROM drive 3026 and the input/output unit such as an IC card drive, and they are connected to the host controller 3010 via the input/output controller 3020. The computer 3000 further includes the ROM 3030 and the legacy input/output unit such as the keyboard 3042, and they are connected to the input/output controller 3020 via the input/output chip 3040.

The CPU 3012 acts pursuant to the program stored in the ROM 3030 and the RAM 3014, and controls respective units by them. The graphic controller 3016 acquires image data generated by the CPU 3012 on a frame buffer or the like provided in the RAM 3014 or in itself, and causes the image data to be displayed on the display device 3018.

The communication interface 3022 communicates with other electronic devices via the network. The hard disk drive 3024 stores the program and data to be used by the CPU 3012 in the computer 3000. The DVD-ROM drive 3026 reads a program or data from the DVD-ROM 3001, and provides the program or data to the hard disk drive 3024 via the RAM 3014. The IC card drive reads the program and data from the IC card, and/or writes the program and data to the IC card.

The ROM 3030 stores a boot program, etc. executed by the computer 3000 at the time of activation, and/or a program dependent on the hardware of the computer 3000. The input/output chip 3040 may connect various input/output units to the input/output controller 3020, via a parallel port, a serial port, a keyboard port, a mouse port, etc.

A program is provided by a computer-readable storage medium like the DVD-ROM 3001 or the IC card. A program is read from the computer-readable storage medium, and installed in the hard disk drive 3024, the RAM 3014 or the ROM 3030, which are the examples of computer-readable storage media, and then executed by the CPU 3012. Information processing described in these programs are read by the computer 3000, and results in cooperation between the program and the above-described various types of hardware resources. An apparatus or method may be configured by realizing an operation or processing of information pursuant to the use of the computer 3000.

For example, if a communication is executed between the computer 3000 and an external device, the CPU 3012 may execute the communication program loaded on the RAM 3014 to instruct communication processing, based on the processing described in the communication program, to the communication interface 3022. The communication interface 3022 reads, under the control by the CPU 3012, the transmission data stored in the transmission buffer region provided in a storage medium such as a RAM 3014, a hard disk drive 3024, a DVD-ROM 3001 or an IC card, sends the transmission data so read to the network, or writes the received data received from the network into a reception buffer region etc. provided on the storage medium.

Also, the CPU 3012 may make all or the necessary portion of a file or a database stored in an external storage medium such as a hard disk drive 3024, a DVD-ROM drive 3026 (DVD-ROM 3001), or an IC card be able to be read by the RAM 3014, to accordingly execute various types of processing to the data on the RAM 3014. Then, the CPU 3012 may write back the processed data into an external storage medium.

Various types of programs, data, table and various types of information such as a database may be stored in a storage medium to receive information processing. The CPU 3012 may execute, against the data read from the RAM 3014, various types of processing, including various types of operations designated by an instruction sequence of a program, which are described throughout this disclosure, an information processing, a condition judgment, a conditional branch, an unconditional branch, information search/replacement, etc., and write back the result to the RAM 3014. Moreover, the CPU 3012 may search information in a file in a storage medium, in a database, etc. For example, if multiple entries, each having an attribute value of a first attribute associated with an attribute value of a second attribute, are stored in the storage medium, the CPU 3012 may search an entry matching with the condition in which the attribute value of the first attribute is designated from among said multiple entries, read the attribute value of the second attribute stored in said entry, and thereby acquire the attribute value of the second attribute associated with the first attribute which satisfies the predefined condition.

A program or a software module explained above may be stored in a computer 3000 or in a computer-readable storage medium in proximity to the computer 3000. Furthermore, a storage medium such as a hard disk or a RAM provided within a server system connected to a dedicated communication network or the Internet can be used as a computer-readable storage medium, to thereby provide the above-described program to the computer 3000 via the network.

While the embodiments of the present invention have been described, the technical scope of the invention is not limited to the above-described embodiments. It is apparent to persons skilled in the art that various alterations and/or improvements can be added to the above-described embodiments. Also, to the extent not being technically inconsistent, matters explained about given embodiment can be applied to other embodiments. Moreover, each component may have features similar to those of other components which have the same name and have the different numerals. It is also apparent from the description of the scope of the claims that the embodiments added with such alterations or improvements can be included in the technical scope of the invention.

The actions, procedures, steps, and stages of each process performed by an apparatus, system, program, and method shown in the claims, embodiments, or diagrams can be executed in any order as long as the order is not indicated by “prior to,” “before,” or the like and as long as the output from a previous process is not used in a later process. Even if the action flow is described using phrases such as “first” or “next” in the claims, embodiments, or diagrams, it does not necessarily mean that the process must be performed in this order.

### EXPLANATION OF REFERENCES

10 communication network, 20 vehicle, 30 user, 32 communication terminal, 34 wearable terminal, 40 facility, 42 activity history acquisition section, 100 navigation system, 110 vehicle control system, 120 assistance server, 220 drive section, 230 body composition sensor, 240 communication section, 250 input/output section, 260 control section, 320 communication control section, 330 input/output control section, 340 drive control section, 350 route search section, 360 user information acquisition section, 420 vehicle information acquisition section, 430 user information acquisition section, 440 storage section, 450 navigation assistance section, 460 control event detection section, 470 control instruction generation section, 522 vehicle information storage section, 524 user information storage section, 532 exercise information storage section, 542 map information storage section, 544 road information storage section, 546 facility information storage section, 548 parking information storage section, 622 start point determination section, 624 destination determination section, 632 candidate point determination section, 634 getting-off point determination section, 642 vehicle route determination section, 644 user route determination section, 720 condition determination section, 722 target determination section, 724 area condition determination section, 730 extraction section, 740 selection section, 862 terminal abnormality detection section, 864 vital abnormality detection section, 866 arrival detection section, 872 alert instruction generation section, 874 move instruction generation section, 875 notification section, 876 wait instruction generation section, 960 control section, 3000 computer, 3001 DVD-ROM, 3010 host controller, 3012 CPU, 3014 RAM, 3016 graphic controller, 3018 display device, 3020 input/output controller, 3022 communication interface, 3024 hard disk drive, 3026 DVD-ROM drive, 3030 ROM, 3040 input/output chip, 3042 keyboard

What is claimed is:

1. An information processing system, comprising:
a first destination information acquisition section for acquiring a first destination information indicating a first destination which is a destination of a user;
a physical information acquisition section for acquiring a physical information indicating a physical feature of the user;
a candidate point determination section for determining a candidate point of a second destination which is a destination of a moving body for conveying the user, based on (i) a position of the first destination indicated by the first destination information, and (ii) a physical feature of the user indicated by the physical information;
wherein the first destination and the second destination are different from each other.

2. The information processing system according to claim 1, wherein:
the physical feature includes at least one of a gender, an age, a body height, a body weight, a muscle mass, a body fat percentage, an ideal body weight, an ideal muscle mass, an ideal body fat percentage, a current health state, a presence/absence of a disorder or an injury, a level of a disorder or an injury, a clinical history, a medical history, a presence/absence of exercise experience, and a level of exercise experience.

3. The information processing system according to claim 1, wherein:
the candidate point determination section determines the candidate point for the second destination, based on (i) a position of the first destination indicated by the first destination information, and (ii) a target value of an energy equivalent amount to be consumed by a physical activity if the user moves from the second destination to the first destination by means of the physical activity of the user without utilizing the moving body.

4. The information processing system according to claim 3, wherein:
the candidate point determination section includes a target value determination section for determining the target value, based on an input related to the target value by the user.

5. The information processing system according to claim 3, wherein:
the candidate point determination section includes a target value determination section for determining the target value, based on the physical feature of the user indicated by the physical information.

6. The information processing system according to claim 5, wherein:
the candidate point determination section includes a category information acquisition section for acquiring a category information indicating a category of a facility positioned in the first destination or a category of a service provided in the facility, and
the target value determination section is configured to determine the target value, based on the category of the facility or the category of the service indicated by the category information.

7. The information processing system according to claim 5, wherein:
the physical information includes information indicating at least one of a current value of a body weight, an age, and a gender,
the candidate point determination section includes:
a distance condition determination section for determining a condition related to a distance between the candidate point for the second destination and the first destination, based on (i) a target value of the energy equivalent amount, and (ii) at least one of the current value of the body weight, the age and the gender of the user indicated by the physical information, and
a mode information acquisition section for acquiring a moving mode information indicating a moving mode when the user moves from the second destination to the first destination;
the distance condition determination section determines a condition related to the distance, based on (i) a target value of the energy equivalent amount, (ii) at least one of the current value of the body weight, the age and the gender of the user indicated by the physical information, and (iii) the moving mode indicated by the moving mode information.

8. The information processing system according to claim 7, wherein:
the candidate point determination section further includes a time information acquisition section for acquiring a desired time information indicating a time at which the user desires to arrive at the second destination; and
the distance condition determination section is configured to determine a condition related to the distance, based on (i) a target value of the energy equivalent amount, (ii) at least one of the current value of the body weight, the age and the gender of the user indicated by the physical information, and (iii) the time indicated by the desired time information.

9. The information processing system according to claim 7, wherein:
the candidate point determination section further includes a road information acquisition section for acquiring road information indicating a road surface status or an altitude profile of roads in a periphery of the first destination; and
the distance condition determination section is configured to determine a condition related to the distance, based on (i) a target value of the energy equivalent amount, (ii) at least one of the current value of the body weight, the age and the gender of the user indicated by the physical information, and (iii) at least one of the road surface status of the road and the altitude profile indicated by the road information.

10. The information processing system according to claim 7, wherein:
the candidate point determination section includes:
an extraction section for referring to a stop location information storage section for storing a stop location information indicating a position of a stop location which is a location at which the moving body can stop so as to extract the stop location whose distance from the first destination satisfies the condition related to the distance determined by the distance condition determination section,
a selection section for selecting at least one of the stop location extracted by the extraction section as the candidate point for the second destination.

11. The information processing system according to claim 10, wherein the selection section is configured to:
acquire, for each of the stop locations extracted by the extraction section, information indicating at least one of a distance of a pathway from the stop location to the first destination, a road surface status of the pathway, and an altitude profile of the pathway;
estimate an energy equivalent amount to be consumed if the user moves from the stop location to the first destination by means of a physical activity of the user, based on at least one of the distance of the pathway, the road surface status of the pathway, and the altitude profile of the pathway; and
select at least one of the stop locations extracted by the extraction section as the candidate point for the second destination, based on an estimated value of the energy equivalent amount.

12. The information processing system according to claim 1, further comprising:
a selection information acquisition section for acquiring user selection information indicating that one of the candidate points for the second destination determined by the candidate point determination section has been selected by the user,
a second destination determination section for determining the candidate point for the second destination indicated by the user selection information as the second destination, and
a sending section for sending information indicating a pathway from the second destination to the first destination to the user's communication terminal.

13. The information processing system according to claim 1, further comprising:
a getting-off detection section for detecting that the user has got off the moving body at the second destination,
a judgment section for judging whether or not a distance between the user's communication terminal and the moving body is within a predefined range, and
an alert determination section for determining to issue an alert to the user, if the judgment section judges that a distance between the communication terminal of the user and the moving body is within a predefined range, after passing a predefined time after the getting-off detection section detects that the user's getting off.

14. The information processing system according to claim 1, wherein
the moving body includes an automatic operation function or a remote operation function, the moving body further including:
a vital information acquisition section for acquiring vital information of the user during a period when the user moves from the second destination to the first destination,
an abnormality detection section for detecting a possibility of potential occurrence of an abnormality to a body of the user, or a possibility of actual occurrence of an abnormality to a body of the user, based on the vital information acquired by the vital information acquisition section; and
an emergency move determination section for determining to move the moving body to a position of the user if the abnormality detection section detects a sign of the abnormality or an occurrence of the abnormality.

15. The information processing system according to claim 14, further comprising:
a notification determination section for determining to notify to a predefined emergency notification destination if the abnormality detection section detects a sign of the abnormality or an occurrence of the abnormality.

16. The information processing system according to claim 1, wherein:
the moving body includes an automatic operation function or a remote operation function, the moving body further including:
a following movement determination section for determining to move the moving body in a manner following after the user so that a distance between the user and the moving body falls within a predefined range, during a period when the user moves from the second destination to the first destination.

17. The information processing system according to claim 1, wherein:
the moving body includes an automatic operation function or a remote operation function, the moving body further including:
a user position detection section for detecting a position of the user,
an arrival detection section for detecting an arrival of the user to the first destination, based on the position of the user detected by the user position detection section, and
a wait determination section for determining to move the moving body to a wait location of the moving body if the arrival detection section detects an arrival of the user.

18. A health management system for managing a health of a user of a vehicle, comprising:
a user profile management section for storing a physical information of the user, and
a vehicle control section for setting a destination of the vehicle, based on the physical information of the user stored in the user profile management section.

19. A non-transitory computer-readable storage medium in which a program for making a computer execute an information processing method is stored, the information processing method including:
acquiring first destination information, by the computer, for acquiring first destination information indicating a first destination which is a destination of a user,
acquiring physical information, by the computer, for acquiring physical information indicating a physical feature of the user, and
determining a candidate point, by the computer, for determining a candidate point for a second destination which is a destination of a moving body conveying the user, based on (i) a position of the first destination indicated by the first destination information, and (ii) the physical feature of the user indicated by the physical information;
wherein the first destination and the second destination are different from each other.

20. An information processing method, comprising:
acquiring first destination information, by a computer, for acquiring first destination information indicating a first destination which is a destination of a user,
acquiring physical information, by the computer, for acquiring physical information indicating a physical feature of the user, and
determining a candidate point, by the computer, for determining a candidate point for a second destination which is a destination of a moving body conveying the user, based on (i) a position of the first destination indicated by the first destination information, and (ii) a physical feature of the user indicated by the physical information;
wherein the first destination and the second destination are different from each other.
